(19) United States
(12) Patent Application Publication
(10) Pub. No.: US 2015/0201831 A1
(43) Pub. Date: Jul. 23, 2015

(54) Portable perimeter for static multi-point synchronous thresholding screening and single-point thresholding detection

(71) Applicant: TIANJIN BHY PHOTOELECTRIC TECHNOLOGY CO., LTD., Tianjin (CN)

(72) Inventors: Zhongjiang He, Tianjin (CN); Jichun Yang, Tianjin (CN); Shuo Liu, Tianjin (CN); Peng Li, Tianjin (CN); Guojiang Gao, Tianjin (CN); Zheng Lv, Tianjin (CN)

(21) Appl. No.: 14/004,904

(22) PCT Filed: Sep. 17, 2012
(86) PCT No.: PCT/CN12/81455

§ 371 Date: Sep. 12, 2013

(30) Foreign Application Priority Data
Aug. 31, 2012 (CN) 201210319741.7
Aug. 31, 2012 (CN) 201220443081.9

(51) Int. Cl. A61B 3/024; G02B 26/08; G02B 27/14; A61B 3/00

(52) U.S. Cl. CPC A61B 3/024; A61B 3/0008; G02B 26/0833; G02B 27/14

(57) ABSTRACT

A portable perimeter for static multi-point synchronous thresholding screening and single-point thresholding detection is provided, comprising: an IPC (industrial personal computer); an LED optical source; an optical splitter; a visual field stimulator; a monitor; a printer; and a transponder; wherein the IPC is connected with the LED optical source, the optical splitter, the monitor, the printer and the transponder; wherein the visual field stimulator is hemispherical, four stimulating holes are provided on the visual field stimulator in such a manner that four beams split out by the optical splitter from a light from the LED optical source respectively pass through the four stimulating holes for forming an optical stimulating signal; wherein the stimulating holes are respectively provided in a GHA15 area, a GHB15 area, a GHC15 area and a GHD15 area according to a GH center 30 degrees visual field partition nomenclature.

### CROSS REFERENCE OF RELATED APPLICATION

This is a U.S. National Stage under 35 U.S.C 371 of the International Application PCT/CN2012/081455, filed Sep. 17, 2012, which claims priority under 35 U.S.C. 119(a-d) to CN 201210319741.7, filed Aug. 31, 2012 and CN 201220443081.9, filed Aug. 31, 2012.

### BACKGROUND OF THE PRESENT INVENTION

1. Field of Invention

The present invention relates to technology fields of clinical medicine and medical instrument, and more particularly to a portable perimeter for static multi-point synchronous thresholding screening and single-point thresholding detection.

2. Description of Related Arts

A perimetry measures visual functions of visual fields of different parts. Clinically, the perimetry is usually provided by a perimeter, and two eyes are examined respectively. The perimeter is an essential auxiliary diagnosis instrument for a department of ophthalmology.

Since the first computer perimeter was published, the visual function of each examined point is indicated by light thresholding sensitivity dB. Since the first microcomputer perimeter (Octopus) was published in 1971, almost a hundred kinds of the microcomputer perimeters have been developed.

As far as stimulation point sources are concerned, there are only three kinds of the microcomputer perimeters: projection type, optical fiber type and LED (light emitting diode) type. Because light intensity of the stimulation point sources of the last two types are various, only the projection perimeters (such as Octopus and Humphery) are recognized. But the projection perimeter is difficult to be popularized because of the large size, the high price, complicated operations and inconvenience to carry; especially, the projection perimeter does not adapt to screening normal population so that visual field defects hidden in the normal population are hard to find.

### SUMMARY OF THE PRESENT INVENTION

An object of the present invention is to provide a portable perimeter with rapid and accurate visual field examination function for static multi-point synchronous thresholding screening and single-point thresholding detection in such a manner that the above shortcomings are overcome.

Accordingly, in order to accomplish the above objects, the present invention provides a portable perimeter for static multi-point synchronous thresholding screening and single-point thresholding detection, comprising:

an IPC (industrial personal computer);

an LED optical source;

an optical splitter;

a visual field stimulator;

a monitor;

a printer; and

a transponder;

wherein the IPC is connected with the LED optical source, the optical splitter, the monitor, the printer and the transponder;

wherein the visual field stimulator is hemispherical, four stimulating holes are provided on the visual field stimulator in such a manner that four beams split out by the optical splitter from a light from the LED optical source respectively pass through the four stimulating holes for forming an optical stimulating signal;

wherein the stimulating holes are respectively provided in a GHA15 area, a GHB15 area, a GHC15 area and a GHD15 area according to a GH center 30 degrees visual field partition nomenclature.

Preferably, the stimulating holes are respectively provided in geometrical centers of the GHA15 area, the GHB15 area, the GHC15 area and the GHD15 area.

Preferably, a radius of the visual field stimulator is 300 mm.

Preferably, the optical splitter comprises:

a DMD (Digital Micromirror Device) comprising four micromirrors;

a convergent lens;

a reflection mirror; and

a shaping lens;

wherein the light from the LED optical source is transmitted to the convergent lens through an optical fiber, then the light is transmitted to the DMD by the reflection mirror, the light is split into the four beams by the four micromirrors, and four beams are transmitted to the stimulating holds on an inner surface of the visual field stimulator after being shaped by the shaping lens for forming the optical stimulation signal.

Preferably, a shutter device is provided on each the micromirror, the shutter device is connected to the IPC through a control circuit.

Preferably, a clear aperture of the micromirror is Goldmann III class.

The stimulating holes of the present invention are respectively provided in the GHA15 area, the GHB15 area, the GHC15 area and the GHD15 area according to the GH center 30 degrees visual field partition nomenclature; the light from the LED optical source is split into the four beams by the optical splitter, and the four beams respectively pass through the four stimulating holes for forming the optical stimulating signal; then luminance of the beams through the four stimulating holes are completely adjusted to the same by the IPC, the LED optical source and the optical splitter; besides, the stimulating holes can be controlled to synchronously generate age-related normal value stimulus in such a manner that rapidest static multi-point thresholding screening with age-related normal values can be provided synchronously, and single-point thresholding detection can also be provided so that visual field defects of glaucoma and neurological ophthalmology diseases can be found accurately.

Furthermore, the present invention has a small size, a low price as well as simple operations, and the present invention is convenient to carry and easy to be popularized. The present invention can be utilized in hospitals of different classes and especially adapts to screening visual fields of normal population.

These and other objectives, features, and advantages of the present invention will become apparent from the following detailed description, the accompanying drawings, and the appended claims.

### BRIEF DESCRIPTION OF THE DRAWINGS

FIG. 1 is a sketch view of a visual field stimulator according to a preferred embodiment of the present invention.

FIG. 2 is a sketch structure view of an optical splitter according to the preferred embodiment of the present invention.

FIG. 3 is a schematic drawing of a GH center 30 degrees visual field partition nomenclature according to the preferred embodiment of the present invention.

FIG. 4 is a diagram of visual field detection according to the preferred embodiment of the present invention.

Reference numbers of elements: 1—convergent lens, 2—reflection mirror, 3—DMD, 4—shaping lens, 5—visual field stimulator, 6—stimulating holes, 7—observing hole.

### DETAILED DESCRIPTION OF THE PREFERRED EMBODIMENT

Referring to the drawings, a portable perimeter for static multi-point synchronous thresholding screening and single-point thresholding detection according to a preferred embodiment of the present invention is illustrated, comprising:

an IPC;

an LED optical source;

an optical splitter;

a visual field stimulator;

a monitor;

a printer; and

a transponder;

wherein the IPC is connected with the LED optical source, the optical splitter, the monitor, the printer and the transponder;

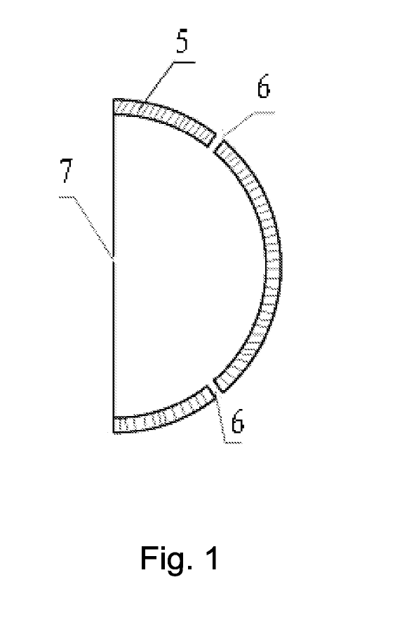

referring to the FIG. 1, wherein the visual field stimulator 5 is hemispherical, four stimulating holes 6 are provided on the visual field stimulator in such a manner that four beams split out by the optical splitter from a light from the LED optical source respectively pass through the four stimulating holes 6 for forming an optical stimulating signal, and when staring into the visual field stimulator 5 through an observing hole 7 (provided on a center of the hemispherical visual field stimulator 5), eyes will be optically stimulated;

wherein the stimulating holes 6 are respectively provided in a GHA15 area, a GHB15 area, a GHC15 area and a GHD15 area according to a GH center 30 degrees visual field partition nomenclature; the four areas are the most sensitive areas for glaucomatous visual field defects, and are distributed in four different quadrants in such a manner that hemianopia and quadrantanopsia can be detected by a department of neurology.

Preferably, a radius of the visual field stimulator 5 is 300 mm.

Preferably, a distance between the observing hole 7 and a center of an inner surface of the visual field stimulator 5 is 300 mm, that is to say the observing hole 7 is provided on the center of the hemispherical visual field stimulator 5.

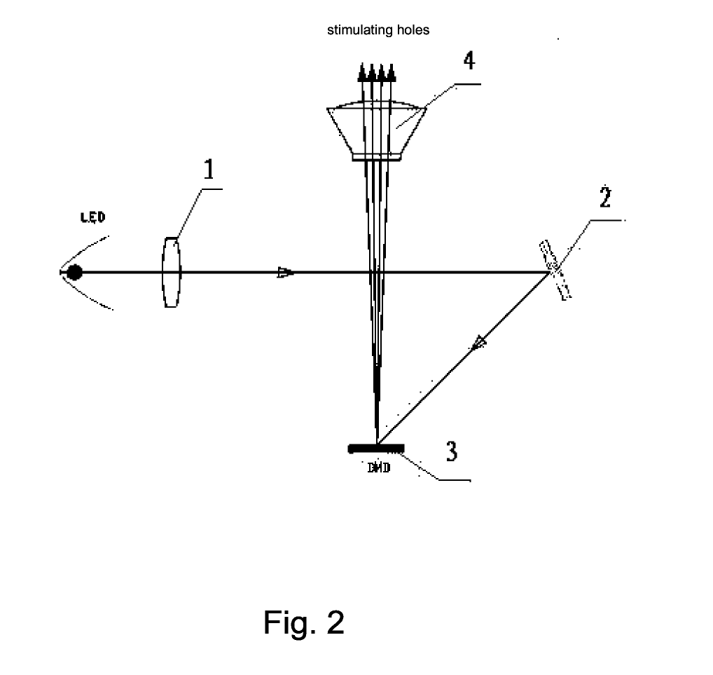

Referring to the FIG. 2, the optical splitter is provided outside the hemispherical surface of the visual field stimulator 5, comprises:

a DMD 3 comprising four micromirrors;

a convergent lens 1;

a reflection mirror 2; and

a shaping lens 4;

wherein the light from the LED optical source (supposing it is an LED) is transmitted to the convergent lens 1 through an optical fiber, then the light is transmitted to the DMD 3 by the reflection mirror 2, the light is split into the four beams by the four micromirrors, and four beams are transmitted to the stimulating holds 6 on the inner surface of the visual field stimulator 5 after being shaped by the shaping lens 4 for forming the optical stimulation signal.

Preferably, a clear aperture of the micromirror is Goldmann III class.

Preferably, a shutter device is provided on each the micromirror, the shutter device is connected to the IPC through a control circuit for replacing an expensive optical gate control device and a prism device.

When providing static multi-point synchronous rapid thresholding screening, the four shutter devices are all opened at the same time; when providing static single-point thresholding detection, only the shutter device corresponding to a detection point is opened, the other shutter devices are all closed in such a manner that the four stimulating holes are respectively utilized for providing static single-point automatic quantitative thresholding detection.

According to the preferred embodiment of the present invention, luminance of the LED optical source is adjusted by a software algorithm. The IPC communicates with a driving circuit of the LED optical source through a R232 port, and drives the LED optical source with quick pulse width modulation (QPWM) mode, wherein a control resolution ratio is 65,535 class. An LED driving current with large range and high accuracy can be controlled by the IPC cooperating with slip resistance tuning and fast switching.

According to the preferred embodiment of the present invention, the four areas with the stimulating holes are worldwide recognized as the most sensitive areas for glaucomatous visual field defects, wherein the areas are well chosen from the areas with eccentricities of 5-25 degrees (in a Bjerrum area) according to a rank of 482 cases of the visual field defects caused by glaucoma during 10 years.

According to reports of the 482 cases of the glaucoma during 1987˜1996, scotomata with a level equal to or higher than 2 in areas 1˜30 of A, B, C and D quadrants are ranked and counted, a result is: for the GHA15 area, the GHB15 area, the GHC15 area and the GHD15 area, numbers of the scotomata with the level equal to or higher than 2 are respectively 462/482=95.8%; 466/482=96.7%; 468/482=97% and 469/482=97.3%. The eye with no scotoma in the four areas is called a zero eye.

The visual field defects of the glaucoma and neurological ophthalmology diseases can be found rapidly and accurately in visual field detection with the most sensitive four areas.

The GH center 30 degrees visual field partition nomenclature is illustrated as follows (wherein the G refers to Greve, a chairman of International Vision Field Symposium; and the H refers to HE, Zhongjiang):

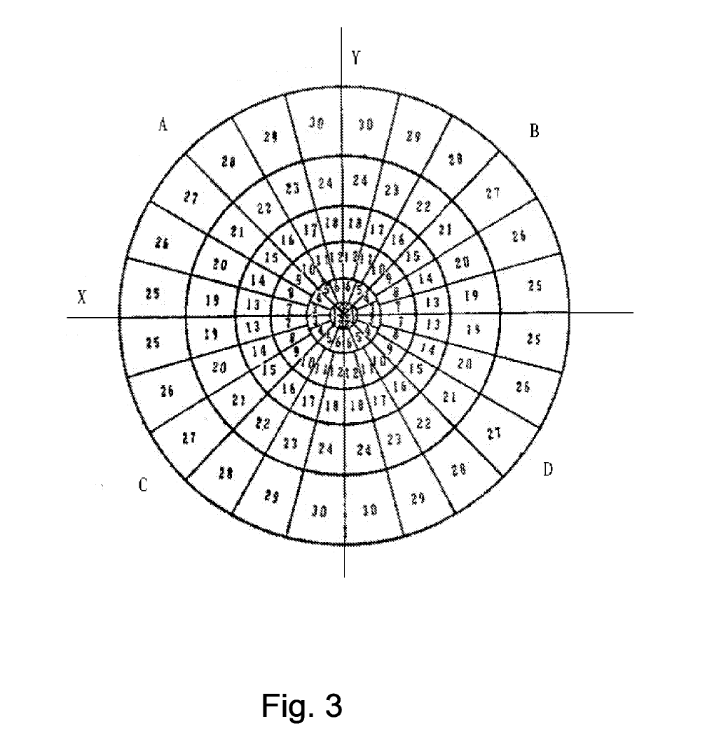

referring to the FIG. 3, the center point O of the inner surface of the visual field stimulator is a cross point; a horizontal axis X and a vertical axis Y are crossed for forming a coordinate, then the cross point O is taken as a center of a circle, and an upper left area, a lower left area, an upper right area and a lower right area are respectively named A, B, C and D quadrant; six concentric circles are drawn with the O as the center and eccentricities of 2, 5, 10, 15, 22 and 30; circle sector areas with the eccentricity of 2 in each quadrant are divided into two areas with central angles of 45 degrees; the circle sector areas with the eccentricity of 2˜5 in each quadrant are divided into four areas with the central angles of 22.5 degrees; the circle sector areas with the eccentricity of 5˜10, 10˜15, 15˜22 and 22˜30 in each quadrant are respectively divided into six areas with the central angles of 15 degrees, and each quadrant is totally divided into 30 areas, area codes are marked from near to far, the areas from horizontal to vertical are marked in an order of 1, 2, . . . and 30 in such a manner that the center 30 degrees visual field can be accurately named with quadrant names (A, B, C, D), eyes difference (OD or OS) and area codes (1-30); the stimulating holes are respectively provided in the GHA15 area, the GHB15 area, the GHC15 area and the GHD15 area.

Preferably, the stimulating holes are respectively provided in geometrical centers of the GHA15 area, the GHB15 area, the GHC15 area and the GHD15 area.

The hemianopia and the quadrantanopsia can be detected by the department of neurology because the four stimulating holes are the most sensitive areas for the glaucomatous visual field defects, and are distributed in four different quadrants; the luminance of the beams through the four stimulating holes are completely adjusted to the same by the LED optical source and the optical splitter with a help of the IPC in such a manner that a disadvantage of that the luminance of the stimulating holes of an LED perimeter can not be adjusted to the same is overcome; besides, the stimulating holes can be controlled to synchronously generate age-related normal value stimulus for providing static multi-point synchronous thresholding screening and single-point thresholding detection so that the visual field defects of glaucoma and neurological ophthalmology diseases can be found accurately and rapidly.

For filling a hemispherical perimeter with a radius of 330 mm with Goldmann III class stimulating points, 375,000 the stimulating points are needed. Averagely, the conventional perimeter in the world has 200 stimulating points. The present invention just need the four stimulating holes provided on the hemispherical visual field stimulator for satisfying clinical requirements of the glaucoma and the department of ophthalmology.

Furthermore, the four stimulating holes can synchronously generate age-related normal value stimulus, and if an examinee responses, the examinee is normal; more than 80% of normal people can be defined as normal within 20 sec; if the examinee doesn't response, the four stimulating holes can synchronously increase or decrease 4 dB to the stimulus for defining the examinee as abnormal or suspected abnormal; the thresholding detection of the four points can be finished within 1 min.

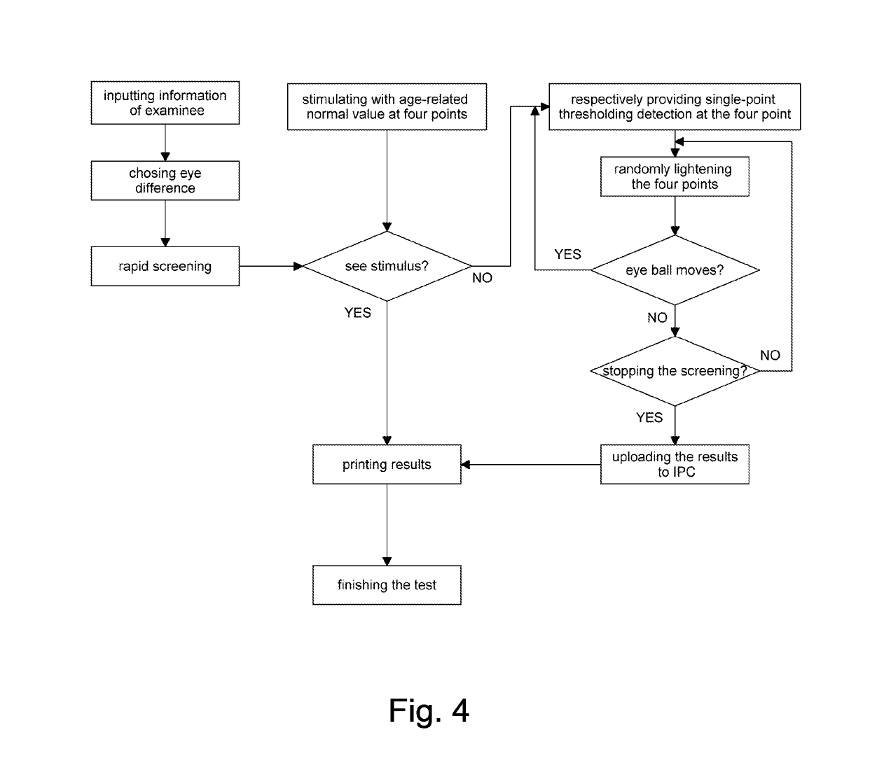

Referring to the FIG. 4, the IPC, as a main controller, is mainly for realizing man-machine interaction; an examiner can provide different testing program according to a condition of the examinee and printing information of the examinee as well as testing results by the printer; the examiner can also control the luminance by communicating with the LDE optical source and analyze as well as show the testing results according to a rapid screening policy of the glaucoma.

When testing the examinee, the information of the examinee is inputted through the IPC, and the eyes difference are chosen according to the information; the IPC sends a testing signal to the LED optical source and provides the corresponding testing program; the examinee receives the optical stimulating signal from the four stimulating holes provided on the inner surface of the visual field stimulator through the observing hole, and during receiving, the monitor checks activity states of eye balls of the examinee at the same time; checking results are instantly fed back to the IPC by the transponder, and are stored by the IPC and (or) printed by the printer.

67 confirmed diagnoses of the glaucoma (75 eyes) have been tested from March, 2006 to September, 2007, wherein 37 male patients and 30 female patients with ages of 14˜79 are tested, an average age is 59.7±10.6. Comparative analysis is provided on the present invention and the Octopus perimeter in a qualitative aspect, a quantitative aspect and a testing time for one eye. Comparing results are as follows:

1. qualitative analysis: according to the Octopus perimeter, a sensitivity and a specificity of the present invention is respectively 92.5% and 86.4%.

2. specificity analysis: an average sensitivity correlation coefficient r1 and an average defect correlation coefficient r2 of the present invention and the Octopus perimeter are respectively 0.869 and 0.865.

3. an average of testing time for one eye of the present invention is 48.65 s, the testing time for one eye of the Octopus perimeter is 141.64 s (t=26.534, P<0.01).

It can thus be seen that four-point thresholding testing according to the present invention adapts to visual field screening of the glaucoma.

The four areas are the most sensitive areas chosen from the Bjerrum areas which are worldwide recognized as the most sensitive areas for the glaucomatous visual field defects; and rapid screening is provided with the age-related normal value at the same time; the four areas are distributed in the four different quadrants in such a manner that the hemianopia and the quadrantanopsia can be detected by the department of neurology, the visual field defects can be detected with 1 min and the thresholding testing can be finished within the same time. Besides, the present invention has a small size, a low price as well as simple operations, and especially adapts to large-scale visual fields screening of normal population.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1-6. (canceled)

7. A portable perimeter for static multi-point synchronous thresholding screening and single-point thresholding detection, comprising:
an IPC (industrial personal computer);
an LED optical source;
an optical splitter;
a visual field stimulator;
a monitor;
a printer; and
a transponder;
wherein said IPC is connected with said LED optical source, said optical splitter, said monitor, said printer and said transponder;
wherein said visual field stimulator is hemispherical, four stimulating holes are provided on said visual field stimulator in such a manner that four beams split out by said optical splitter from a light from said LED optical source respectively pass through said four stimulating holes for forming an optical stimulating signal;
wherein said stimulating holes are respectively provided in a GHA15 area, a GHB15 area, a GHC15 area and a GHD15 area according to a GH center 30 degrees visual field partition nomenclature.

8. The portable perimeter, as recited in claim 7, wherein said stimulating holes are respectively provided in geometrical centers of said GHA15 area, said GHB15 area, said GHC15 area and said GHD15 area.

9. The portable perimeter, as recited in claim 7, wherein a radius of said visual field stimulator is 300 mm.

10. The portable perimeter, as recited in claim 8, wherein a radius of said visual field stimulator is 300 mm.

11. The portable perimeter, as recited in claim 7, wherein said optical splitter comprises:
a DMD (Digital Micromirror Device) comprising four micromirrors;
a convergent lens;
a reflection mirror; and
a shaping lens;
wherein said light from said LED optical source is transmitted to said convergent lens through an optical fiber, then said light is transmitted to said DMD by said reflection mirror, said light is split into said four beams by said four micromirrors, and four beams are transmitted to said stimulating holds on an inner surface of said visual field stimulator after being shaped by said shaping lens for forming said optical stimulation signal.

12. The portable perimeter, as recited in claim 8, wherein said optical splitter comprises:
a DMD (Digital Micromirror Device) comprising four micromirrors;
a convergent lens;
a reflection mirror; and
a shaping lens;
wherein said light from said LED optical source is transmitted to said convergent lens through an optical fiber, then said light is transmitted to said DMD by said reflection mirror, said light is split into said four beams by said four micromirrors, and four beams are transmitted to said stimulating holds on an inner surface of said visual field stimulator after being shaped by said shaping lens for forming said optical stimulation signal.

13. The portable perimeter, as recited in claim 9, wherein said optical splitter comprises:
a DMD (Digital Micromirror Device) comprising four micromirrors;
a convergent lens;
a reflection mirror; and
a shaping lens;
wherein said light from said LED optical source is transmitted to said convergent lens through an optical fiber, then said light is transmitted to said DMD by said reflection mirror, said light is split into said four beams by said four micromirrors, and four beams are transmitted to said stimulating holds on an inner surface of said visual field stimulator after being shaped by said shaping lens for forming said optical stimulation signal.

14. The portable perimeter, as recited in claim 10, wherein said optical splitter comprises:
a DMD (Digital Micromirror Device) comprising four micromirrors;
a convergent lens;
a reflection mirror; and
a shaping lens;
wherein said light from said LED optical source is transmitted to said convergent lens through an optical fiber, then said light is transmitted to said DMD by said reflection mirror, said light is split into said four beams by said four micromirrors, and four beams are transmitted to said stimulating holds on an inner surface of said visual field stimulator after being shaped by said shaping lens for forming said optical stimulation signal.

15. The portable perimeter, as recited in claim 11, wherein a shutter device is provided on each said micromirror, said shutter device is connected to said IPC through a control circuit.

16. The portable perimeter, as recited in claim 12, wherein a shutter device is provided on each said micromirror, said shutter device is connected to said IPC through a control circuit.

17. The portable perimeter, as recited in claim 13, wherein a shutter device is provided on each said micromirror, said shutter device is connected to said IPC through a control circuit.

18. The portable perimeter, as recited in claim 14, wherein a shutter device is provided on each said micromirror, said shutter device is connected to said IPC through a control circuit.

19. The portable perimeter, as recited in claim 11, wherein a clear aperture of said micromirror is Goldmann III class.

20. The portable perimeter, as recited in claim 12, wherein a clear aperture of said micromirror is Goldmann III class.

21. The portable perimeter, as recited in claim 13, wherein a clear aperture of said micromirror is Goldmann III class.

22. The portable perimeter, as recited in claim 14, wherein a clear aperture of said micromirror is Goldmann III class.

23. The portable perimeter, as recited in claim 15, wherein a clear aperture of said micromirror is Goldmann III class.

24. The portable perimeter, as recited in claim 16, wherein a clear aperture of said micromirror is Goldmann III class.

25. The portable perimeter, as recited in claim 17, wherein a clear aperture of said micromirror is Goldmann III class.

26. The portable perimeter, as recited in claim 18, wherein a clear aperture of said micromirror is Goldmann III class.
